(19) United States
(12) Patent Application Publication
(10) Pub. No.: US 2023/0398242 A1
(43) Pub. Date: Dec. 14, 2023

(54) Fibroblast Activation ImmunoPET for Detection of Fibrosis Activity

(71) Applicant: Wisconsin Alumni Research Foundation, Madison, WI (US)

(72) Inventors: Reinier Hernandez, Madison, WI (US); Aaron LeBeau, Madison, WI (US); Chris Massey, Madison, WI (US); Zachary Rosenkrans, Madison, WI (US); Joseph Gallant, Madison, WI (US)

(21) Appl. No.: 18/332,457

(22) Filed: Jun. 9, 2023

Related U.S. Application Data
(60) Provisional application No. 63/351,260, filed on Jun. 10, 2022.

(51) Int. Cl. A61K 51/10

(52) U.S. Cl. CPC A61K 51/1075

(57) ABSTRACT

This disclosure relates to fibroblast activation immune-Pet for detection of fibrosis activity.

### CROSS-REFERENCE TO RELATED APPLICATIONS

This application claims priority to U.S. provisional application No. 63/351,260, filed Jun. 10, 2022, the disclosure of which is expressly incorporated by reference herein.

### SEQUENCE LISTING

This application contains a Sequence Listing submitted as an electronic text file named “22-0924-US_SequenceListing_ST26.xml,” having a size in bytes of 8.05 kb, and created on Jun. 8, 2023. The information contained in this electronic file is hereby incorporated by reference in its entirety.

### BACKGROUND OF THE DISCLOSURE

### Field of Invention

This disclosure relates to fibroblast activation immunopet for detection of fibrosis activity, for example, pulmonary fibrosis activity.

### Technical Background

Several inflammatory processes present similar features to fibrosis (e.g., pulmonary fibrosis) in high-resolution CT. However, there are no reliable methods to predict who will develop fibrosis whether due to being at high-risk from genetic predisposition or life style or due to receiving treatment for a medical condition, for example, radiotherapy for lung cancer.

Fibroblast activation protein (FAP) is a protease that is overexpressed during the pathogenic activation of fibroblasts. Activated fibroblasts are associated with extracellular matrix remodeling and initiation of the deposition of extracellular matrix associated with cancer and fibrosis in tissues. Many research tool grade antibodies exist to study this protein, but they may not be the correct species or have the specificity needed to be useful diagnostic reagents or cell targeting delivery vehicles.

In addition, there is a lack of methods with high specificity for detecting fibrinogenesis in patients. For example, current methodologies for detecting pulmonary fibrosis activity are imperfect and do not enable clinicians to predict progression and response to treatments, such as radiotherapy and chemotherapy. Computed tomography (CT) is the standard way of detecting lung fibrosis but can only detect late disease when significant fibrotic burden and lung function impairment are present (and has limitations noted above). Therapeutic interventions at that point are ineffective.

Therefore, there is a need for improved detection of fibroblast activation preceding fibrinogenesis that can sensitively predict progression into fibrosis earlier than current methods, providing opportunity for more timely interventions.

### SUMMARY OF THE DISCLOSURE

This disclosure provides compositions and methods for detecting fibroblast activation preceding fibrinogenesis. Such compositions and methods can be used to detect onset and progression of fibrosis in patients. Further, such compositions and methods can be used to tailor therapeutic intervention in patients, for example, such as dosing and timing of administration of radiotherapy and chemotherapy.

In a first aspect, the present disclosure provides a humanized antibody or antigen binding fragment thereof specific for fibroblast activation protein (FAP). The humanized antibody or antigen binding fragment thereof includes a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3.

In one embodiment of the first aspect, the heavy chain variable region comprises greater than 95% and optionally less than 100% of the amino acid sequence of SEQ ID NO: 8, and wherein the light chain variable region comprises greater than 95% and optionally less than 100% of the amino acid sequence of SEQ ID NO: 7. In one embodiment of the first aspect, the antibody is an IgG1. In one embodiment of the first aspect, the antibody is a full antibody, a chimeric antibody, a CDR-grafted antibody, or a recombinant antibody. In one embodiment of the first aspect, the antigen binding fragment is a Fab, Fab′, F(ab′)2, Fabc, or Fv. In one embodiment of the first aspect, the humanized antibody or antigen binding fragment thereof is conjugated with a chelating agent. In one embodiment, the chelating agent is deforoxamine. In some embodiments, the deforoxamine is bound with a radionuclide for PET/CT imaging. In some embodiments, the radionuclide is Zr-89.

In a second aspect, the present disclosure provides a pharmaceutical composition including a pharmaceutically effect amount of the humanized antibody or antigen binding fragment thereof of any one of the preceding first aspect and embodiments thereof; and a pharmaceutically acceptable carrier or diluent.

In a third aspect, the present disclosure provides a method of detecting development of fibrotic tissue in a subject. The method includes a) administering a humanized antibody or antigen binding fragment thereof specific for fibroblast activation protein (FAP), the humanized antibody or antigen binding fragment thereof comprising i) a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3, ii) a chelating agent, and iii) a radionuclide for PET/CT imaging. The method further includes detecting the radionuclide in the subject by PET/CT.

In one embodiment of the third aspect, the location of detection of the radionuclide indicates the location of developing fibrotic tissue. In one embodiment of the third aspect, the developing fibrotic tissue is associated with cancer. In one embodiment, the cancer is lung cancer. In one embodiment of the third aspect, the developing fibrotic tissue is caused by radiotherapy and/or chemotherapy.

In a fourth aspect, the present disclosure provides a method of treating cancer a subject in need thereof including a) administering a first dose of radiotherapy and/or chemotherapy to the subject; b) administering a humanized antibody or antigen binding fragment thereof specific for fibroblast activation protein (FAP) to the subject, the humanized antibody or antigen binding fragment thereof comprising i) a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3, ii) a chelating agent, and iii) a radionuclide for PET/CT imaging; c) detecting a level of the radionuclide at a treatment site in the subject by PET/CT; and d) administering a second dose of radiotherapy and/or chemotherapy to the subject if the level of radionuclide detected in the subject does not indicate the development of fibrotic tissue at the treatment site.

In one embodiment of the fourth aspect, the radionuclide is Zr-89.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. ⁸⁹Zr-labeled anti-FAP PET/CT in lung SBRT. Mice received 100 Gy of Stereotactic Body RT (SBRT) in the right lung. Two weeks after irradiation, mice were administered an ⁸⁹Zr-labeled anti-FAP monoclonal antibody and serial PET/CT scans were acquired at 24, 72, and 144 h post-injection of the radiotracer. Radiotracer uptake was colocalized with the irradiated areas of the lung indicating in situ FAP expression. Of note, fibrosis in this model occurs between 4 and 8 weeks post-irradiation.

FIGS. 2A-2E. Non-invasive detection of pulmonary fibrosis using a Zr-89 radiolabeled fibroblast activation protein (FAP) targeting antibody (⁸⁹Zr-Df-huB12 IgG). (2A) Competitive and (2B) saturation binding assays evaluated the affinity of huB12 IgG to human FAP with CWR-R1^{FAP} cancer cells. (2C) Representative axial PET/CT images showed high lung uptake of ⁸⁹Zr-Df-huB12 mAb in bleomycin groups compared to control groups when administered 6 d or 13 d post instillation. Quantitative analysis of lung uptake based on (2D) whole lung volume of interest analysis and (2E) ex vivo biodistribution analysis found increase uptake in the lungs of mice administered bleomycin compared to controls. Statistical analysis compared to bleomycin to control at identical time following instillation (* P<0.05, ** P<0.01).

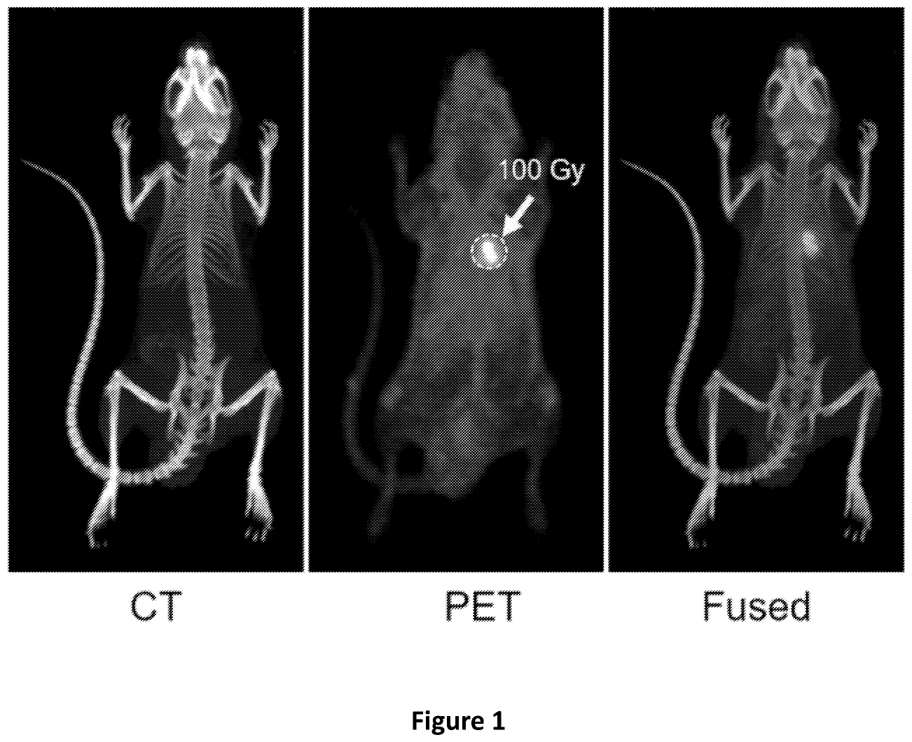

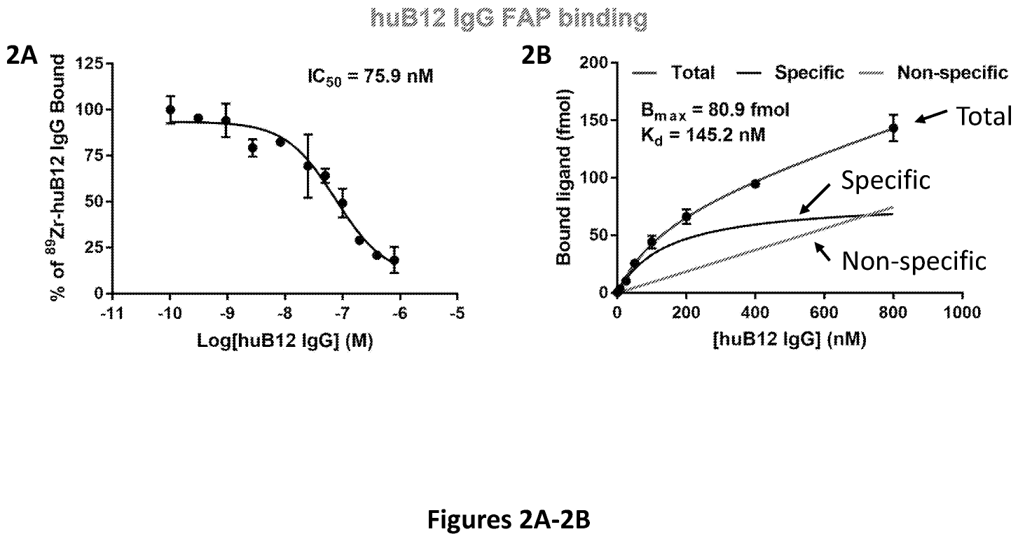

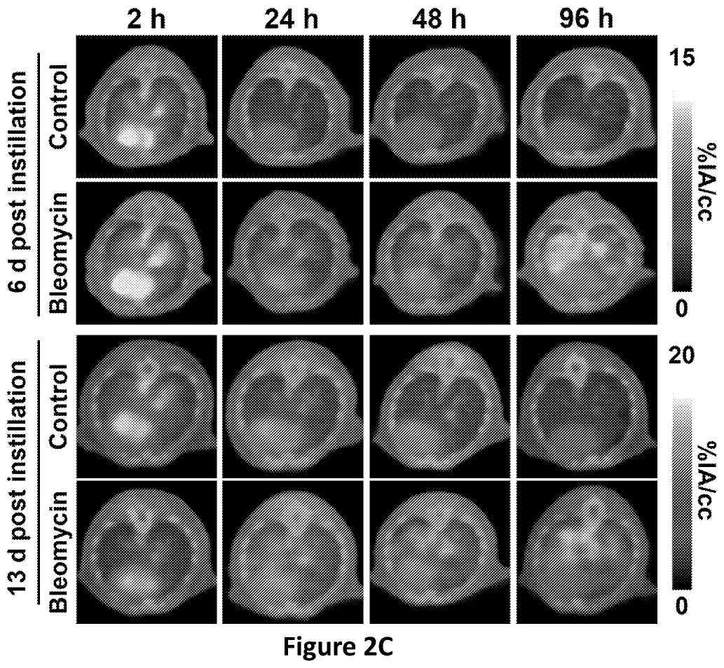

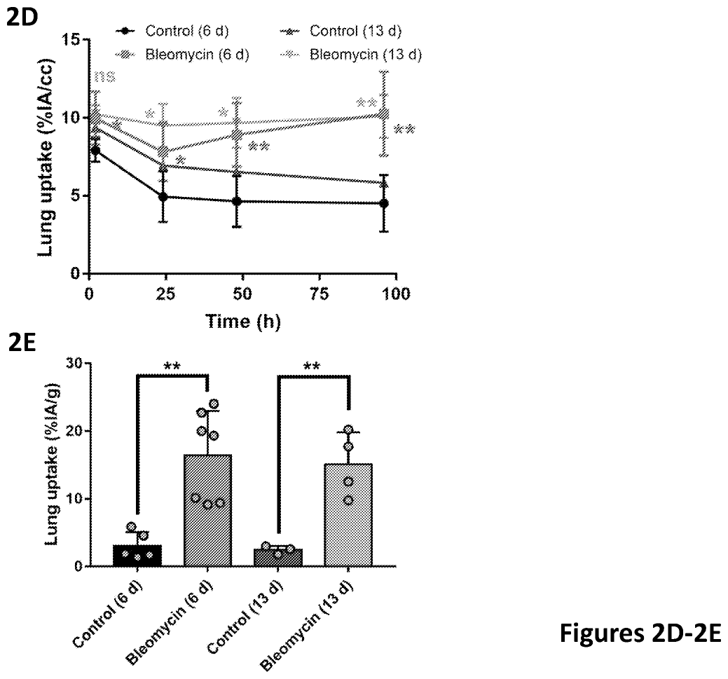

### DETAILED DESCRIPTION

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Patient” or “subject” refers to a warm blooded animal such as a mammal, preferably a human, which is afflicted with, or has the potential to be afflicted with one or more diseases and disorders described herein.

In view of the present disclosure, the methods and compositions described herein can be configured by the person of ordinary skill in the art to meet the desired need.

### Overview

Disclosed herein are novel, cross-species antibodies directed to fibroblast activation protein (FAP). The antibodies underwent modifications to be humanized and were subsequently labeled with radionuclides for PET imaging (the process can be referred to as “immune-Pet”). The resulting antibodies have high specificity in labeling active fibrosis in animal models for fibrosis (e.g., lung fibrosis) and various cancers. The antibodies can detect early fibrotic activity in tissue which enables early diagnosis and treatment of fibrosis.

In some embodiments, the present disclosure is directed to humanized antibodies or antigen binding fragment thereof that specifically bind FAP. For example, contemplated humanized antibodies or antigen binding fragment thereof include a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3.

Additional antibodies contemplated for use herein can be found in U.S. application Ser. No. 16/778,977, which is incorporated by reference herein in its entirety for all purposes.

In some embodiments, contemplated humanized antibodies or antigen binding fragment thereof include a heavy chain variable region that has greater than 95% and less than 100% of the amino acid sequence of SEQ ID NO: 8 and a light chain variable region that has greater than 95% and less than 100% of the amino acid sequence of SEQ ID NO: 7. In some embodiments, the humanized antibodies or antigen binding fragment thereof is (or is derived from) an IgG antibody, for example, an IgG1 antibody. However, any isotype of antibody is contemplated herein.

In some embodiments, contemplated humanized antibodies can be a full antibody, a chimeric antibody, a CDR-grafted antibody, or a recombinant antibody. In some embodiments, contemplated antigen binding fragment can be a Fab, Fab′, F(ab′)2, Fabc, or Fv.

In some embodiments, contemplated humanized antibodies or antigen binding fragment thereof can be conjugated with a chelating agent. Any number or type of chelating agent is contemplated for use herein. In some embodiments, the chelating agent is deforoxamine.

In some embodiments, contemplated humanized antibodies or antigen binding fragment thereof are labeled with one or more imaging agents that permit detection of the humanized antibodies or antigen binding fragment thereof when administered internally to a subject. For example, contemplated imaging agents include those that are detectable by positron emission tomography (PET) and/or computed tomography (CT). In certain preferred embodiments, the imaging agents are radionuclides. In some embodiments, a contemplated radionuclide is Zr-89.

In other embodiments, contemplated humanized antibodies or antigen binding fragment thereof can be conjugated with therapeutic molecules for delivery to developing fibrotic tissues. Examples of such therapeutic molecules include those that can prevent, slow, or inhibit fibrinogenesis.

### Compositions

In some embodiments, pharmaceutical compositions including a pharmaceutically effect amount of the humanized antibody or antigen binding fragment thereof described herein are contemplated. For example, a pharmaceutical composition can include a sufficient amount of the humanized antibody or antigen binding fragment thereof described herein and a pharmaceutically acceptable carrier or diluent. Pharmaceutically effect amounts of contemplated humanized antibodies or antigen binding fragments thereof can readily be determined by those of skill in the art.

### Methods

In some embodiments, the present disclosure is directed to methods of imaging fibrotic tissues using the inventive imaging agents described herein (e.g., radionuclide-labeled humanized anti-FAP antibodies).

For example, contemplated herein are methods of detecting development of fibrotic tissue in a subject. Such methods can includes a) administering a humanized antibody or antigen binding fragment thereof specific for fibroblast activation protein (FAP), the humanized antibody or antigen binding fragment thereof comprising i) a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3, ii) a chelating agent, and iii) a radionuclide for PET/CT imaging. The method further includes detecting the radionuclide in the subject by PET/CT.

Any route of administration is contemplated herein, such as by inhalation or parenteral, enteral, and/or topical administration.

It is contemplated herein that the location of detection of the radionuclide indicates a location of developing fibrotic tissue. Such locations can be indicative of developing fibrotic tissue associated with cancer (e.g., lung cancer or any other type of cancer that gives rise to developing fibrotic tissue). In addition, the location of developing fibrotic tissue can be merely associated with the treatment of a disease at the location. For example, the developing fibrotic tissue can be caused by radiotherapy and/or chemotherapeutic treatment of a tumor.

In other embodiments, methods of treating cancer a subject are contemplated. For example, such methods can include a) administering a first dose of radiotherapy and/or chemotherapy to the subject; b) administering a humanized antibody or antigen binding fragment thereof specific for fibroblast activation protein (FAP) to the subject, the humanized antibody or antigen binding fragment thereof comprising i) a heavy chain variable region comprising heavy chain CDR1, CDR2, and CDR3 and a light chain variable region comprising light chain CDR1, CDR2, and CDR3, wherein heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3, ii) a chelating agent, and iii) a radionuclide for PET/CT imaging; c) detecting a level of the radionuclide at a treatment site in the subject by PET/CT; and d) administering a second dose of radiotherapy and/or chemotherapy to the subject if the level of radionuclide detected in the subject does not indicate the development of fibrotic tissue at the treatment site. In such embodiments, any radionuclide is contemplated for use. In one example, the radionuclide can be Zr-89.

In other embodiments, it is contemplated herein that patients at high risk for developing fibrosis (e.g., due to genetic predisposition, organ transplant, and/or lifestyle choices) can be monitored using the humanized antibodies and antigen binding fragments thereof, and pharmaceutical compositions including such agents, via PET/CT when receiving treatment for various conditions. For example, patients undergoing radiation therapy (e.g., lung radiation therapy, etc.) could be scanned to detect early onset of radiation pneumonitis and fibrosis cause by the treatment. In this way, it is contemplated that the imaging agents and methodologies described herein can be combined with fibrosis-causing therapies (without limitation) to enable clinicians to be able to monitor and even modulate dosing and/or frequency of such treatments to improve treatment outcomes.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Detecting Pulmonary Fibrosis Activity Through Immuno-PET Imaging of Fibroblast Activation Protein

Introduction

The absence of non-invasive biomarkers to sensitively detect interstitial lung diseases, such as idiopathic pulmonary fibrosis (IPF), is a major burden limiting timely clinical diagnosis. During fibrogenesis in IPF, activated fibroblasts are the principal effector cell driving lung extracellular matrix remodeling leading to functional deterioration and ultimately failure. The overexpression of fibroblast activation protein (FAP) on activated fibroblasts is a promising biomarker for disease detection. To this end, the ability of an engineered anti-FAP antibody (huB12 IgG) to visualize in vivo FAP expression in a mouse model of pulmonary fibrosis by PET/CT was assessed.

Methods

A fully humanized huB12 IgG was engineered to target FAP. The affinity of huB12 IgG to human FAP binding was evaluated with competition and saturation binding assays using CWR-R1FAP cancer cells engineered to express FAP. A mouse model of pulmonary fibrosis was established by intratracheal administration of bleomycin (1 U/kg) or saline as control. Pulmonary fibrosis and FAP expression were confirmed histologically through Masson's trichrome staining and immunohistochemistry. huB12 IgG was conjugated with deferoxamine (Df) for Zr-89 radiolabeling (t_{1/2}=78.4 h) and purified using a size exclusion column (⁸⁹Zr-Df-huB12 IgG). Cohorts of male and female mice from bleomycin or saline group were intravenously injected with ˜6.3 MBq (170 μCi) of ⁸⁹Zr-Df-huB12 IgG at 6 d or 13 d post instillation for PET/CT imaging. Serial PET/CT (Inveon microPET/CT) was performed at 2 h, 24 h, 48 h, and 96 h post-injection (p.i.) and reconstructed using an OSEM3D/MAP algorithm. Volume of interest analysis (Inveon Research Workplace) of the whole lung quantified lung density in Hounsfield units (HU) and radiotracer uptake in percent injected activity per cubic centimeter (% IA/cc). Following the final imaging time points, mice were euthanized and tissue uptake was quantified using ex vivo biodistribution studies as percent injected activity per gram of tissue (% IA/g).

Results

Using competitive and saturation binding studies with CWR-R1FAP cells, we determined the IC₅₀, Kd, and receptor to be 75.9 nM, 145.2 nM, and ˜2.3×10⁵ receptors/cell, respectively. CT imaging did not detect differences in whole-lung CT density between mice administered bleomycin or saline at any of the imaging time points. In contrast, lung uptake of ⁸⁹Zr-Df-huB12 IgG in PET/CT was significantly higher in mice administered bleomycin compared to control at 6 d and 13 d post-injury as early as 24 h p.i. ⁸⁹Zr-Df-huB12 IgG lung uptake peaked at 96 h p.i. in bleomycin mice at both 6 d (10.3±2.7% IA/cc) and 13 d post instillation (10.1±1.4% IA/cc), and was 2.2-fold (4.6±2.0% IA/cc) and 1.7-fold (5.8±0.3% IA/cc) higher than respective control groups. Statistically higher lung uptake (P<0.05) of ⁸⁹Zr-Df-huB12 IgG in bleomycin mice compared to control mice occurred at 2 h p.i. onwards at 6 d post instillation (p=0.029 at 2 h p.i., p=0.02 at 24 h p.i., p=0.005 at 48 h p.i., and p=0.003 at 96 h p.i.), and at 24 h p.i. onwards at 13 d post instillation (p=0.027 at 24 h p.i., p=0.021 at 48 h p.i., and p=0.003 at 96 h p.i.). Ex vivo biodistribution at 96 h p.i. confirmed significant higher lung uptake in bleomycin mice compared to control mice when ⁸⁹Zr-Df-huB12 IgG was administered 6 d post instillation (16.4±6.6% IA/g vs 3.1±2.0% IA/g; p=0.002) and 13 d post instillation (15.1±4.87% IA/g vs 2.5±0.6% IA/g; p=0.007).

### CONCLUSION

Immuno-PET targeting FAP is an attractive biomarker for non-invasively detecting early pulmonary fibrosis activity. Based on our findings, we will further investigate ⁸⁹Zr-Df-huB12 IgG PET as a diagnostic tool for pulmonary fibrosis and in other fibrotic diseases.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. description and the appended claims. Although some aspects of the present disclosure can be identified herein as particularly advantageous, it is contemplated that the present disclosure is not limited to these particular aspects of the disclosure.

Furthermore, the disclosure encompasses all variations, combinations, and permutations in which one or more limitations, elements, clauses, and descriptive terms from

Sequence Listing

| SEQ ID | Sequence | Name |
|---|---|---|
| SEQ ID | RASQEISGYLS | LCDR1 or light |
| NO: 1 | | chain CDR1 |
| SEQ ID | AASTLDS | LCDR2 or light |
| NO: 2 | | chain CDR2 |
| SEQ ID | LQYASYPWT | LCDR3 or light |
| NO: 3 | | chain CDR3 |
| SEQ ID | GFTFSSYGMS | HCDR1 or heavy |
| NO: 4 | | chain CDR1 |
| SEQ ID | TINSNGGSTYYPDSVKG | HCDR2 or heavy |
| NO: 5 | | chain CDR2 |
| SEQ ID | DYFDY | HCDR3 or heavy |
| NO: 6 | | chain CDR3 |
| SEQ ID | DIVITQSPSSLSASLGERVS | VL |
| NO: 7 | LTCRASQEISGYLSWLQQKP | |
| | DGTIKLIYAASTLDSGVPKR | |
| | FSGSRSGSDYSLTISSLESE | |
| | DFADYYCLQYASYPWTFGGG | |
| | TKLEIKR | |
| SEQ ID | EVMLVESGGGLVQPGGSLKL | VH |
| NO: 8 | SCAASGFTFSSYGMSWVRQT | |
| | PDKRLELVATINSNGGSTYY | |
| | PDSVKGRFTISRDNAKNTLY | |
| | LQMSSLKSEDTAMYYCAR | |
| | DYFDYWGQGTTLTVSS | |

What is claimed is:

1. A humanized antibody or antigen binding fragment thereof specific for fibroblast activation protein (FAP), comprising a heavy chain variable region comprising heavy chain CDR1, CDR2, and CDR3 and a light chain variable region comprising light chain CDR1, CDR2, and CDR3, wherein
heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3.

2. The humanized antibody or antigen binding fragment thereof of claim 1, wherein the heavy chain variable region comprises greater than 95% and optionally less than 100% of the amino acid sequence of SEQ ID NO: 8, and wherein the light chain variable region comprises greater than 95% and optionally less than 100% of the amino acid sequence of SEQ ID NO: 7.

3. The humanized antibody or antigen binding fragment thereof of claim 1, wherein the antibody is an IgG1.

4. The humanized antibody or antigen binding fragment thereof of claim 1, wherein the antibody is a full antibody, a chimeric antibody, a CDR-grafted antibody, or a recombinant antibody.

5. The humanized antibody or antigen binding fragment thereof of claim 1, wherein the antigen binding fragment is a Fab, Fab′, F(ab′)2, Fabc, or Fv.

6. The humanized antibody or antigen binding fragment thereof of claim 1, wherein the humanized antibody or antigen binding fragment thereof is conjugated with a chelating agent.

7. The humanized antibody or antigen binding fragment thereof of claim 6, wherein the chelating agent is deforoxamine.

8. The humanized antibody or antigen binding fragment thereof of claim 7, wherein the deforoxamine is bound with a radionuclide for PET/CT imaging.

9. The humanized antibody or antigen binding fragment thereof of claim 8, wherein the radionuclide is Zr-89.

10. A pharmaceutical composition, comprising:
a pharmaceutically effect amount of the humanized antibody or antigen binding fragment thereof of any one of the preceding claims; and
a pharmaceutically acceptable carrier or diluent.

11. A method of detecting development of fibrotic tissue in a subject, comprising:
a) administering a humanized antibody or antigen binding fragment thereof specific for fibroblast activation protein (FAP), the humanized antibody or antigen binding fragment thereof comprising
i) a heavy chain variable region comprising heavy chain CDR1, CDR2, and CDR3 and a light chain variable region comprising light chain CDR1, CDR2, and CDR3, wherein
heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3,
ii) a chelating agent, and
iii) a radionuclide for PET/CT imaging; and
b) detecting the radionuclide in the subject by PET/CT.

12. The method of claim 11, wherein the location of detection of the radionuclide indicates the location of developing fibrotic tissue.

13. The method of claim 11, wherein the developing fibrotic tissue is associated with cancer.

14. The method of claim 13, wherein the cancer is lung cancer.

15. The method of claim 11, wherein the developing fibrotic tissue is caused by radiotherapy and/or chemotherapy.

16. A method of treating cancer a subject in need thereof, comprising:
a) administering a first dose of radiotherapy and/or chemotherapy to the subject;
b) administering a humanized antibody or antigen binding fragment thereof specific for fibroblast activation protein (FAP) to the subject, the humanized antibody or antigen binding fragment thereof comprising
i) a heavy chain variable region comprising heavy chain CDR1, CDR2, and CDR3 and a light chain variable region comprising light chain CDR1, CDR2, and CDR3, wherein
heavy chain CDR1 comprises the amino acid sequence of SEQ ID NO: 4, heavy chain CDR2 comprises the amino acid sequence of SEQ ID NO: 5, heavy chain CDR3 comprises the amino acid sequence of SEQ ID NO: 6, light chain CDR1 comprises the amino acid sequence of SEQ ID NO: 1, light chain CDR2 comprises the amino acid sequence of SEQ ID NO: 2, and light chain CDR3 comprises the amino acid sequence of SEQ ID NO: 3,
ii) a chelating agent, and
iii) a radionuclide for PET/CT imaging;
c) detecting a level of the radionuclide at a treatment site in the subject by PET/CT; and
d) administering a second dose of radiotherapy and/or chemotherapy to the subject if the level of radionuclide detected in the subject does not indicate the development of fibrotic tissue at the treatment site.

17. The method of claim 16, wherein the radionuclide is Zr-89.


SEQUENCE LISTING (8 sequences)

SEQ ID NO: 1 - protein, 11 aa, Synthetic construct
RASQEISGYLS

SEQ ID NO: 2 - protein, 7 aa, Synthetic construct
AASTLDS

SEQ ID NO: 3 - protein, 9 aa, Synthetic construct
LQYASYPWT

SEQ ID NO: 4 - protein, 10 aa, Synthetic construct
GFTFSSYGMS

SEQ ID NO: 5 - protein, 17 aa, Synthetic construct
TINSNGGSTYYPDSVKG

SEQ ID NO: 6 - protein, 5 aa, Synthetic construct
DYFDY

SEQ ID NO: 7 - protein, 107 aa, Synthetic construct
DIVITQSPSSLSASLGERVSLTCRASQEISGYLSWLQQKPDGTIKLIYAASTLDSGVPKRFSGSRSGSDYSLTISSLESEDFADYYCLQYASYPWTFGGGTKLEIKR

SEQ ID NO: 8 - protein, 114 aa, Synthetic construct
EVMLVESGGGLVQPGGSLKLSCAASGFTFSSYGMSWVRQTPDKRLELVATINSNGGSTYYPDSVKGRFTISRDNAKNTLYLQMSSLKSEDTAMYYCARDYFDYWGQGTTLTVSS